(19) United States
(12) Patent Application Publication
(10) Pub. No.: US 2026/0060792 A1
(43) Pub. Date: Mar. 5, 2026

(54) USE OF PGAM2 IN BREEDING OF BLCATTLE BLACK CATTLE

(71) Applicant: Qingdao Agricultural University, Qingdao (CN)

(72) Inventors: Yajuan DONG, Qingdao (CN); Xiuyuan WANG, Qingdao (CN); Ruili LIU, Qingdao (CN)

(21) Appl. No.: 19/087,226

(22) Filed: Mar. 21, 2025

(30) Foreign Application Priority Data
Sep. 4, 2024 (CN) 202411233930.1

(51) Int. Cl. A61D 19/00; C12N 5/071; C12N 15/113; C12Q 1/533; C12Q 1/6888; G01N 33/50

(52) U.S. Cl. CPC A61D 19/00; C12N 5/0683; C12N 15/1138; C12Q 1/533; C12Q 1/6888; G01N 33/5023; C12N 2310/14; C12N 2310/531; C12N 2503/02; C12N 2510/00; C12Q 2600/124; C12Y 504/02; G01N 2333/99; G01N 2500/10

(57) ABSTRACT

Provided is use of PGAM2 in the breeding of Blcattle black cattle, which belongs to the technical field of molecular biological breeding, in which individuals having a high PGAM2 content are screened as high-quality breeding bulls. In the present disclosure, it is proved by means of experiments that PGAM2 may promote the proliferation and glycolytic metabolism of Sertoli cells to produce lactic acid, an energy substrate required for spermatogenic cell development. It regulates the formation of blood-testis barrier and facilitates the communication between Sertoli cells and germ cells, thereby promoting spermatogenesis. The present disclosure provides a new direction for the breeding of Blcattle black cattle for high quality beef production.

### CROSS-REFERENCE TO RELATED APPLICATION

This patent application claims the benefit and priority of Chinese Patent Application No. 202411233930.1 filed with the China National Intellectual Property Administration on Sep. 4, 2024, the disclosure of which is incorporated by reference herein in its entirety as part of the present application.

### REFERENCE TO SEQUENCE LISTING

A computer readable XML file entitled “GWP20250100143_seqlist”, which was created on Feb. 19, 2025, with a file size of about 19,967 bytes, contains the sequence listing for this application, has been filed with this application, and is hereby incorporated by reference in its entirety.

### TECHNICAL FIELD

The present disclosure belongs to the technical field of molecular biological breeding, and specifically relates to use of PGAM2 in the breeding of Blcattle black cattle.

### BACKGROUND

The information disclosed in the background section aims to enhance the understanding of general background of the present disclosure, and it should not be construed as an acknowledgement or implication in any form that the information constitutes the prior art known to those of ordinary skill in the art.

Fertility is one of the most important economic traits in beef cattle production. With regard to breeding bulls, their reproductive performances are mainly determined by semen quality. In the testis tissue, the normal progress of testis development and spermatogenesis processes is a prerequisite to ensure semen quality. The spermatogenesis process is regulated by a variety of cells in testis tissue, such as Sertoli cells, interstitial cells and spermatogenic cells. Among them, Sertoli cells provide energy and nutrient support for the spermatogenesis process. In the glycolytic metabolism process in Sertoli cells, glucose can be converted into lactic acid and cytokines and hormones are secreted simultaneously, providing energy matrix for the growth and development of germ cells, thereby promoting the spermatogenesis process to be completed smoothly. Sertoli cells can prevent toxic substances from entering spermatogenic cells, and phagocytose and scavenge apoptotic spermatogenic cells and residues due to problems such as nutrient shortage, thereby avoiding spermatogenic damage. Sertoli cells are also a major component of the blood-testis barrier in the testis. The blood-testis barrier separates the germ cell lymphatic system from the circulatory system on the basis of the tight junctions between Sertoli cells, providing immune protection for the spermatogenesis process. The interaction between Sertoli cells can provide morphological support to germ cells, and the Sertoli cells coordinate with the germ cells to regulate the spermatogenesis process. Therefore, maintaining the number and function of Sertoli cells is essential to improve the fertility of breeding bulls.

Phosphoglycerate mutase (PGAM) was first discovered in yeast and later researchers found that it was expressed in a variety of organisms. PGAM is a key enzyme in the glycolytic pathway, and its main function is to catalyze the conversion of 3-phosphoglycerate (3-PG) to 2-phosphoglycerate (2-PG) during glycolysis, participating in other metabolic processes through the conversion balance between 3-PG and 2-PG. Studies have found that there are five subtypes of PGAM, namely PGAM1-5, and PGAM2 is one of them. Studies in cancer cells have shown that PGAM2 plays an important role in the Warburg effect (i.e., increased glycolysis). Studies in muscle cells have shown that deletion of PGAM2 results in loss of actin polymerization sites. In addition, a study by Kondoh et al. demonstrated that when the expression level of PGAM2 was increased by 2 times in mouse embryonic fibroblasts, the mouse embryonic fibroblasts could immortalize and resist ras-induced senescence. Another study on a gene of the same gene family has found that PGAM1 regulates microfilament assembly by directly interacting with ACTA2. Microfilaments constitute F-actin, participate in the formation of the cytoskeleton, and provide attachment sites for blood-testis barrier related proteins, including tight junction proteins, connexin and matrix-specific proteins, making them essential for the function of blood-testis barrier. In addition, microfilaments are involved in the formation of focal adhesions and adhesion belts, and the adhesion belts regulate cell adhesion, mechanical sensing and signals that control cell growth and differentiation. A study of glioma cells by Lu et al. have found that silencing PGAM4 can attenuate the cell viability, proliferation and glycolysis of T98G human brain glioma cells to inhibit tumor growth in vivo, while overexpressing PGAM4 promotes the cell viability, proliferation and glycolysis of U251 human glioblastoma cells by regulating the glycolytic pathway. Cheng et al. found that PGAM5 is highly expressed in liver cancer, and that knockdown of PGAM5 can inhibit tumor growth. The main mechanism is that PGAM5 can bind to and stabilize Bcl-xL and inhibit Bax and cytochrome C mediated apoptotic signals, and thus contributes to the survival of tumor cells.

Blcattle black cattle is the first new germplasm in beef cattle successfully cultivated by somatic cell nuclear transplantation (SCNT) in China. This breed was developed by improving the Luxi yellow cattle and Bohai black cattle using modern biotechiques such as somatic cell cloning combined with conventional breeding and hybridization methods to enable offspring to show a combination of excellent characteristics. The hybrid germplasm fully shows heterosis, retains the beef quality characteristic and some appearance characteristics, and acquires the physiological characteristics of Luxi yellow cattle and Bohai black cattle that adapt to the local environment and geographical conditions. The most notable feature of this breed is its good beef quality, which shows typical marble patterns, also known as “snowflake beef”. The beef is tender and juicy, rich in iron content, low in saturated fatty acid content and high in unsaturated fatty acid content. It possesses a unique flavor, exquisite taste, distinctive texture, and is rich in nutrition, making it highly valuable for beef production. In 2015, the breed was recognized by experts as a new population, and is considered an excellent cattle breed for the production of marbled beef. The fertility of breeding bulls is an important economic trait that directly affects the production performance and reproductive efficiency of cows. Increasing the fertility of breeding bulls can increase the conception rate and the quality of fetuses in cows, thereby increasing the reproductive effect. This provides a feasible way to speed up the breeding of Blcattle black cattle. Therefore, further optimizing the germplasm resources of Blcattle black cattle is of great significance for further popularizing the breed.

### SUMMARY

To address the problems existing in the conventional technology, the present disclosure provides a method for screening high-quality breeding bulls of Blcattle black cattle through PGAM2. PGAM2 plays a role in the regulation of proliferation, apoptosis and glycolytic metabolism in bovine Sertoli cells and the regulation of the actin cytoskeleton in bovine Sertoli cells. It promotes the production of lactic acid to provide energy for germ cell development, regulates the formation of blood-testis barrier, and facilitates communication between Sertoli cells and germ cells, thereby affecting spermatogenesis.

To achieve the above objective, the present disclosure provides the following technical solutions.

Provided is use of phosphoglycerate mutase 2 (PGAM2) gene in the breeding of Blcattle black cattle.

In some embodiments, the use includes screening individuals having a high PGAM2 content as breeding cattle.

In some embodiments, the use includes overexpressing PGAM2 in breeding cattle or breeding cattle testis tissue by using drugs or genetic engineering methods.

Provided is an engineered bovine somatic cell, where the engineered bovine somatic cell is a bovine Sertoli cell in which PGAM2 gene is knocked down, knocked out, or overexpressed.

Provided is a method for preparing the above engineered bovine somatic cell, including the steps of:
(1) isolating Sertoli cell from bovine testis tissue;
(2) transfecting the Sertoli cell with an overexpression plasmid or a siRNA/shRNA plasmid of PGAM2 gene; and
(3) screening Sertoli cells having up regulated or down regulated PGAM2 gene expression.

SiRNAs used the method have the nucleotide sequences selected from the group consisting of SEQ ID NOs: 7 and 8, SEQ ID NOs: 9 and 10, and SEQ ID NOs: 11 and 12.

The above engineered bovine somatic cell can be used for assessing effects of PGAM2 on the proliferation, apoptosis, glycolytic metabolism and actin cytoskeleton of bovine Sertoli cells; or for screening drugs that target PGAM2.

Embodiments of the present disclosure have the following advantages:

In the present disclosure, it is proved through experiments that PGAM2 can regulate the formation of blood-testis barrier and facilitate communication between Sertoli cells and germ cells by regulating the microfilament skeleton of Blcattle black cattle Sertoli cells. It promotes the production of lactic acid and ATP by regulating the proliferation, apoptosis and glycolysis of Sertoli cells, thereby promoting spermatogenesis. In breeding, individuals having a high PGAM2 content should be screened as high-quality breeding bulls of Blcattle black cattle. The present disclosure provides a new direction for the breeding of Blcattle black cattle for high-quality beef production.

### BRIEF DESCRIPTION OF THE DRAWINGS

FIGS. 1A-1B show the volcano plot (FIG. 1A) and PCA plot (FIG. 1B) of DEGs;

FIGS. 2A-2C show GO analysis (FIG. 2A), KEGG enrichment analysis (FIG. 2B) and glycolytic/gluconeogenic signaling pathway (FIG. 2C) of DEGs;

FIG. 3 is a diagram of PGAM2 protein interaction network;

FIGS. 4A-4C show analysis of mRNA (FIG. 4A) and protein (FIGS. 4B-4C) expression patterns of PGAM2 in different tissues;

FIGS. 5A-5C show analysis of mRNA (FIG. 5A) and protein (FIGS. 5B-5C) expressions of PGAM2 in Big testis and Small testis;

FIG. 6 shows identification of Sertoli cells;

FIGS. 7A-7F show the transfection efficiency of PGAM2 plasmids;

FIGS. 8A-8B show the proliferation of Sertoli cells in different treatment groups as detected by EDU;

FIGS. 9A-9D shows detection of mRNA (FIGS. 9A-9B) and protein (FIGS. 9C-9D) of proliferation apoptosis-related genes in different treatment groups;

FIG. 10 shows the effect of PGAM2 gene expression on downstream genes of glycolytic pathway in Sertoli cells;

FIG. 11 shows staining of microfilament skeleton by phalloidin.

### DETAILED DESCRIPTION OF THE EMBODIMENTS

The present disclosure will be further described with reference to examples and accompanying drawings, but the present disclosure is not limited by the examples described below.

### Example 1. Screening of Breeding-Related Differentially Expressed Genes (DEGs) in Blcattle Black Cattle

### 1. Construction of Blcattle Black Cattle Testis Phenotype and Proteome Library

Blcattle black cattle (bulls) were selected from Shandong Zhaofu Animal Husbandry Technology Co., Ltd. The testis tissues were collected from eight healthy Blcattle black cattle (bulls) in similar feeding environment. The testis tissues were divided into two groups according to the weight, long diameter and short diameter, namely Big testis (BT) and Small testis (ST), respectively, with four testis tissues per group. After slaughter, the heart, liver, spleen, lung, kidney and skeletal muscle tissues were collected, placed in 5 mL cryopreservation tubes, and stored in liquid nitrogen for later use.

TABLE 1
Phenotypic data of Blcattle black cattle in BT group and ST group

| | | | Long | Short |
|---|---|---|---|---|
| Group | Weight/kg | Testis weight/g | diameter/cm | diameter/cm |
| BT | 243.67 ± 14.43 | 186.00 ± 32.19^{a} | 10.50 ± 0.87^{a} | 4.50 ± 0.00^{a} |
| ST | 259.33 ± 12.57 | 110.67 ± 6.43^{b} | 9.00 ± 0.00^{b} | 3.67 ± 0.29^{b} |

Notes:
long diameter refers to the ventral longitudinal length of testis, and short diameter refers to the ventral transverse length of testis; no letters in the same column of data indicate insignificant differences (P > 0.05), and different lowercase letters in the same column of data indicate significant differences (P < 0.05).

The testis tissue samples were pre-treated using an iST sample pre-treatment kit (PreOmics®, Germany). The tissues were ground in liquid nitrogen. An appropriate amount of sample was added into, 50 ul of lysis liquid and heated for 10 min at 95° C., 1000 rpm. The sample was cooled to room temperature, then a trypsin digestion buffer was added for shaking incubation for 2 h at 37° C., 500 rpm. The stop buffer was added to terminate the enzymatic hydrolysis reaction. The peptide fragment was desalted using the iST cartridge in the kit, eluted using 2×100 μL of elution buffer, followed by suction drying under vacuum and then stored at −80° C. After that, high PH reverse phase separation and low PH nano-scale High Performance Liquid Chromatography-Tandem Mass Spectrometry (nano-HPLC-MS/MS) analysis (Data-Dependent Acquisition (DDA)-based qualitative database construction) were performed. The raw data were subjected to pooling, analysis and database search by Spectronaut X (Biognosys AG). Finally Data-Independent Acquisition (DIA) was performed.

### 2. Screening of Differentially Expressed Protein

### 2.1 Analysis of Differential Expression

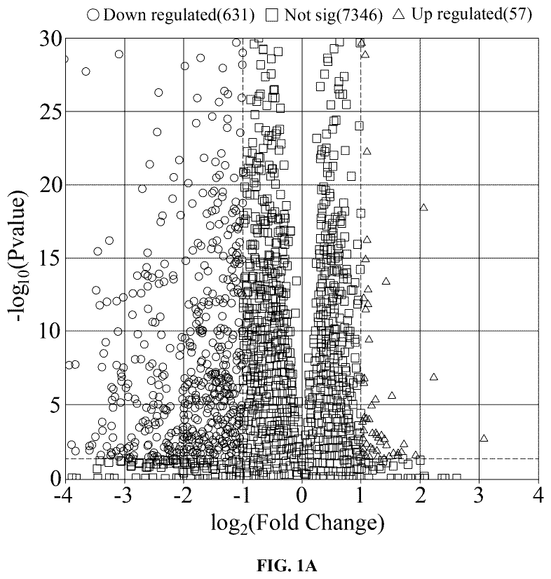

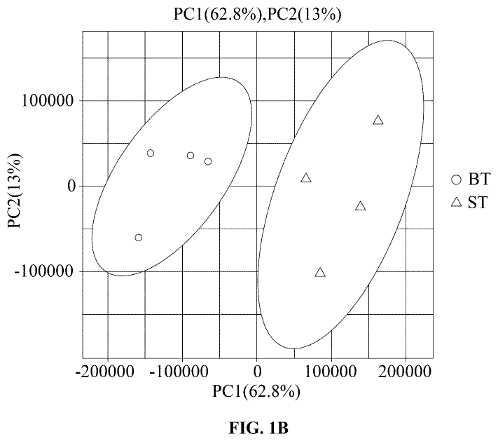

Differentially expressed proteins (DEPs) for BT and ST were determined using DEseq2 software, with P-value ≤0.05 and |Fold Change|≥2 as screening criteria. The analysis of the volcano plot (FIG. 1A) of DEPs demonstrated that there were a total of 688 DEPs between the two groups, with 57 proteins up-regulated and 631 proteins down-regulated in ST. The analysis of the principle component analysis (PCA) plot (FIG. 1B) showed that the four biological samples from the same group were clustered together, indicating that the biological replicates used in the study are of good quality.

### 2.2 Gene Oncology (GO) and Kyoto Encyclopedia of Genes and Genomes (KEGG) Enrichment Analysis

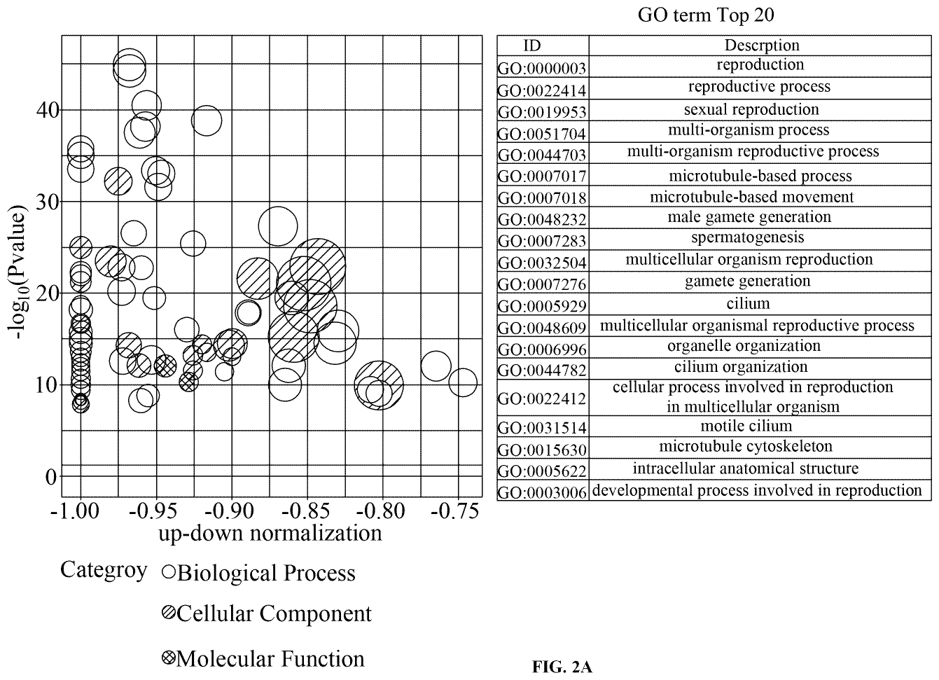

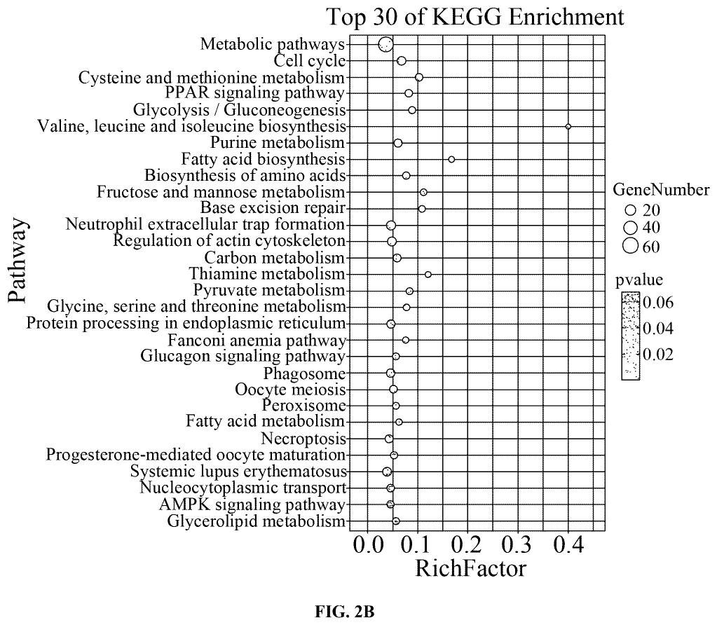

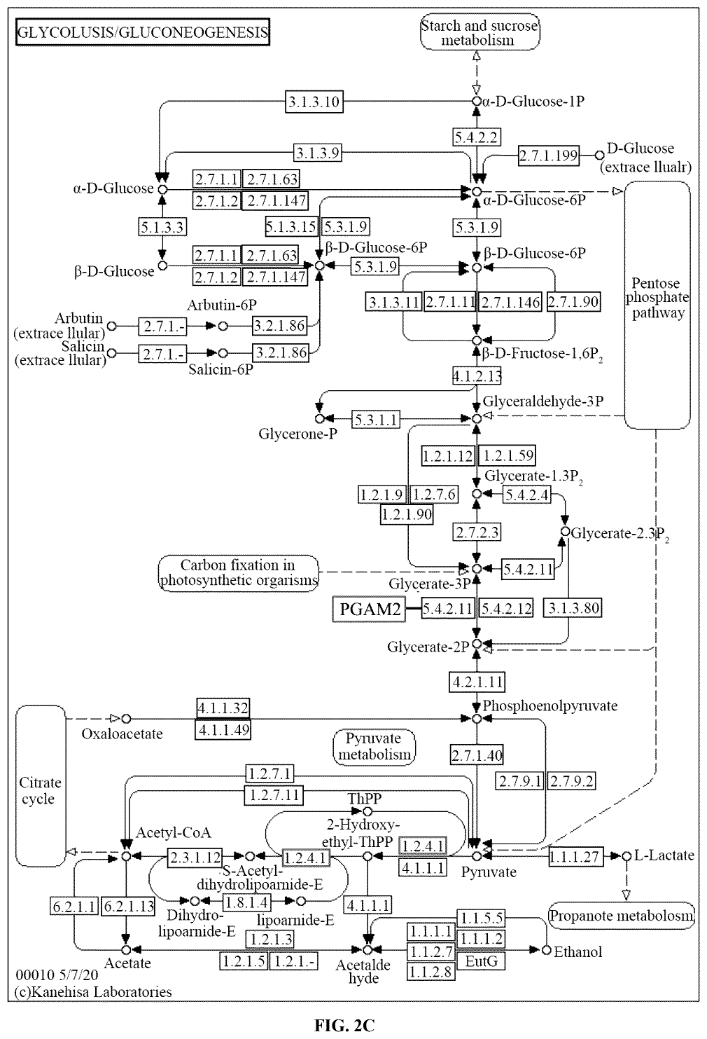

To identify the potential biological functions of DEPs, GO and KEGG enrichment analyses were performed on DEPs. GO terms can be divided into three categories, including: Biological Process (BP), Cellular Component (CC) and Molecular Function (MF). The threshold for significantly enriched GO terms and KEGG pathways was set at P value <0.05. The results in FIGS. 2(A and 2B) showed that DEPs were significantly enriched in biological processes such as sexual reproduction, male gamete production, and spermatogenesis; and DEPs were significantly enriched in cellular components such as cilia, motile cilia, microtubule cytoskeleton and intracellular anatomy. The KEGG enrichment results showed that DEPs were mainly enriched in reproduction-related pathways (Peroxisome Proliferator-Activated Receptor (PPAR) signaling pathway, glycolytic/gluconeogenic signaling pathway, and regulatory signaling pathway of actin cytoskeleton, etc.). By analyzing the glycolytic/gluconeogenic signaling pathway (FIG. 2C), it is found that PGAM2 is capable of catalyzing the conversion of 3-phosphoglycerate (3-PG) to 2-phosphoglycerate (2-PG) during glycolysis to affect downstream gene expression, thereby regulating the production of ATP and lactic acid.

### 3. Functional Analysis of PGAM2 and Identification of Expression Profiles in Tissues

### 3.1 Analysis of Protein Interaction Network

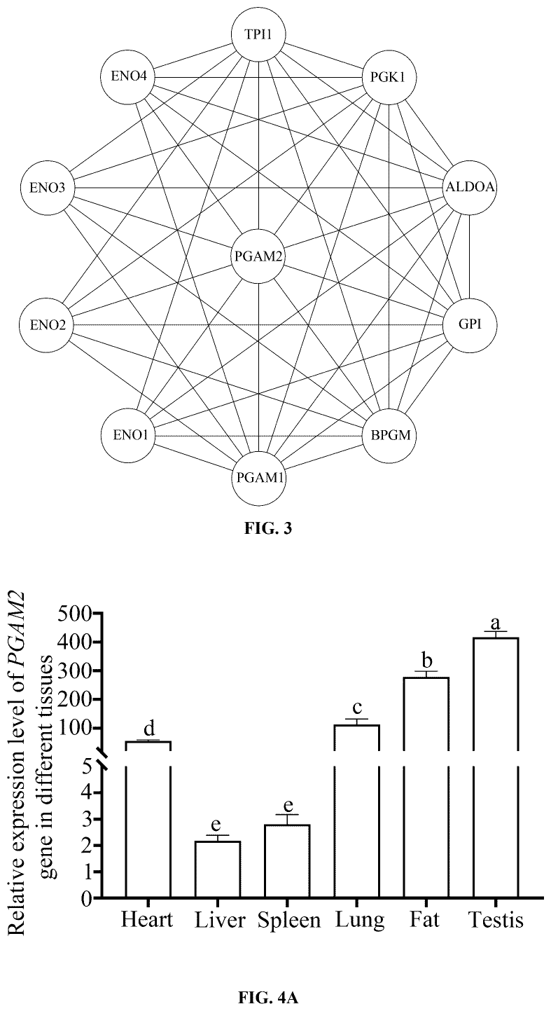

A protein interaction network for PGAM2 was constructed using the STRING database (string-db.org/), and then visually displayed using Cytoscape 6.1 software. The results in FIG. 3 showed that there were 10 proteins that interact directly with PGAM2, namely TPI1, PGK1, PGAM1, GPI, ENO4, ENO3, ENO2, ENO1, BPGM and ALDOA, where the molecular functions of PGAM1, TPI1, ENO1 and ALDOA were related to the proliferation, apoptosis and glycolytic metabolism of Sertoli cells, affecting spermatogenesis. PGAM2 was therefore screened as a candidate gene related to male reproduction.

### 3.2 Identification of Expression Profiles in Different Tissues, Big Testis Tissue and Small Testis Tissue

The testis tissues from BT and ST groups (four testis tissues per group), as well as the heart, liver, spleen, lung and muscle were collected, and stored in liquid nitrogen. The total RNA was extracted using Trizol reagent (Invitrogen®), and cDNA was obtained by reverse transcription. Taking the cDNA as a template, SYBR Green qPCR of PGAM2 gene was performed using primers in Table 2. Glyceraldehyde-3-phosphate dehydrogenase (GAPDH) was used as the internal reference gene, the CT values of each sample were detected, the mean value was calculated, and the relative expression levels of PGAM2 gene in different tissues, Big testis tissue and Small testis tissue were calculated. At the same time, each tissue protein was extracted using RadioImmunoprecipitation Assay (RIPA) lysis buffer, the concentration of the extracted tissue protein was determined using a BCA kit. Using GAPDH as the internal reference gene, the relative expression levels of PGAM2 protein in each tissue were calculated after western blot assay.

TABLE 2
Quantitative polymerase chain reaction
(qPCR) primers for PGAM2 and GAPDH

| | | SEQ ID |
|---|---|---|
| Gene | Primer sequence (5′→3′) | NO: |
| PGAM2 | F: TTTGACATCTGCTACACATCGG | 1 |
| | R: ACTTGTAGTAGGGGTGTTTCTCA | 2 |
| GAPDH | F: GATGCTGGTGCTGAGTATGT | 3 |
| | R: GCAGAAGGTGCAGAGATGAT | 4 |

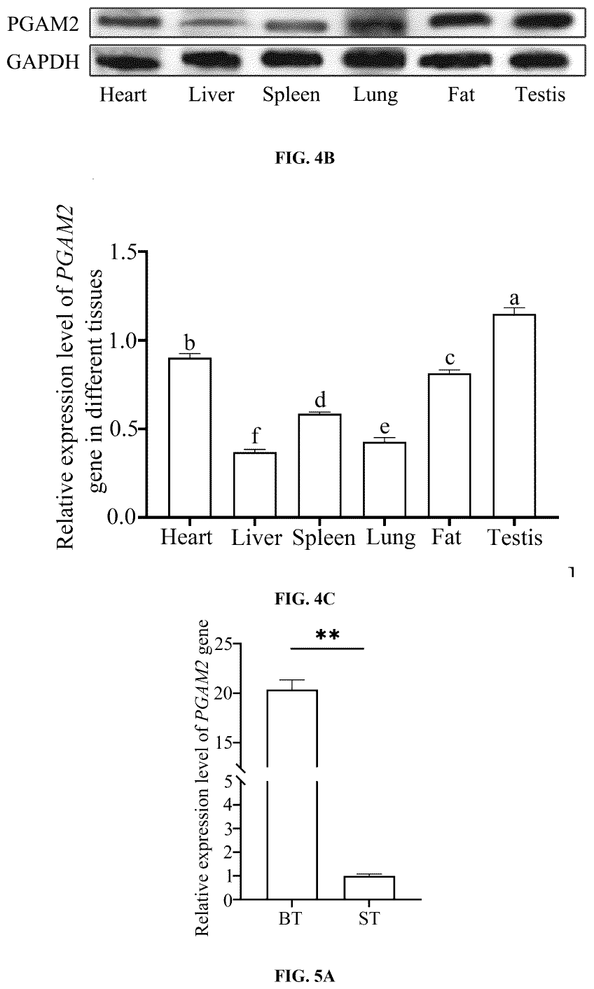

The analysis of mRNA (FIG. 4A) and protein (FIGS. 4B-4C) expressions of PGAM2 in different tissues showed that the PGAM2 gene is expressed to varying degrees in the heart, liver, spleen, lung, fat and testis of Blcattle black cattle, with a significantly higher expression level in the testis than that in the other tissues (P<0.05). The expression level of PGAM2 protein in the testis was the highest, significantly higher than that in the other tissues (P<0.05). The analysis of mRNA (FIG. 5A) and protein (FIGS. 5B-5C) expressions of PGAM2 in Big testis tissue and Small testis tissue (FIGS. 5A-5C) showed that the PGAM2 was expressed in both Big testis and Small testis, with the expression level in Big testis extremely significantly higher than that in Small testis (P<0.01), and the protein expression level was consistent with the gene expression level.

### Example 2 Construction of Sertoli Cells with Differential Expression of PGAM2

### 1. Culture and Identification of Sertoli Cells

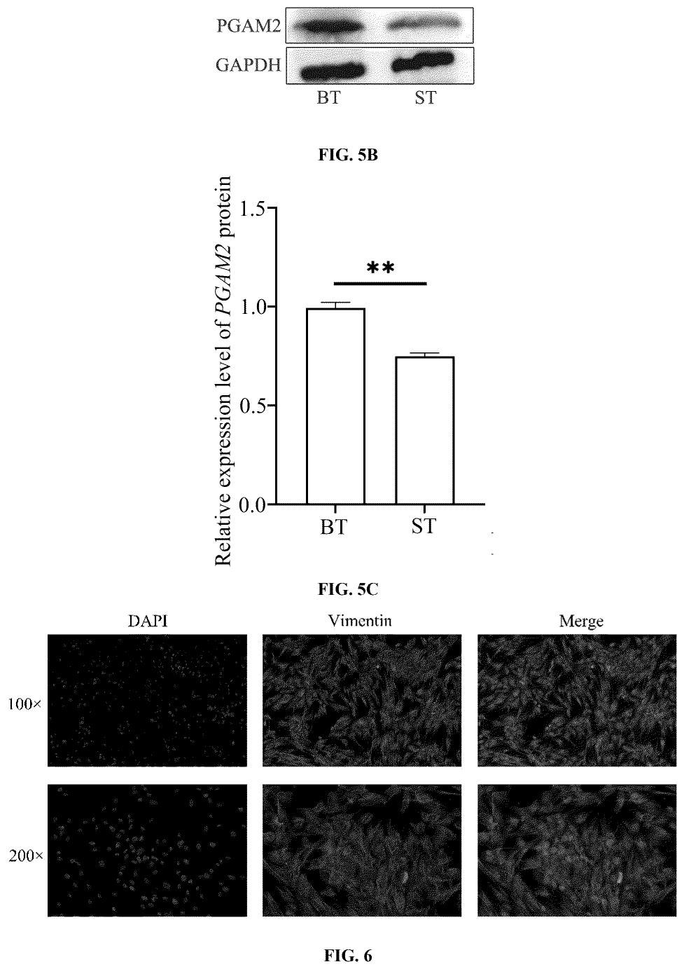

Bovine primary cells were isolated from the testis tissue of newly born Blcattle calf and cultured. These cells were made into cell slides, which were then fixed with 4% paraformaldehyde for 15 min, permeabilized with 0.5% Triton X-100 for 2 h, and blocked with a blocking liquid for 2 h. CD34 antibody was added for incubation overnight at 4° C. A fluorescent secondary antibody was added and incubated for 2 h at 37° C. 4′,6-Diamidino-2-PhenylIndole (DAPI) was added for incubation for 5 min in the dark. The slide was sealed with a Fluoromount-G mounting medium, and Vimentin and DAPI fluorescence signals were collected. The results (FIG. 6) showed that Vimentin, a marker for Sertoli cells, was expressed on the cells, indicating that the isolated cells are Sertoli cells.

2. Construction of Sertoli Cells with Differential Expression of PGAM2

On the basis of the mRNA sequence of PGAM2 (GenBank No. NM_001038111.2), three siRNA sequences as well as a negative control (NC) were designed and synthesized. The sequences are shown in Table 3. The PGAM2 gene was cloned into a vector to construct the PGAM2 overexpression vector PGAM2-OE, and an empty vector NC-OE was used as a control.

TABLE 3
Sequences for constructing Sertoli cells
with differential expression of PGAM2

| | | SEQ ID |
|---|---|---|
| Group | Sequence (5′→3′) | NO: |
| NC siRNA | F: UUCUCCGAACGUGUCACGUTT | 5 |
| | R: TACGUGACACGUUCGGAGAATT | 6 |
| PGAM2 siRNA- | F: GAUGCAGAGCUGAGCGAGATT | 7 |
| 77 | R: UCUCGCUCAGCUCUGCAUCTT | 8 |
| PGAM2 siRNA- | F: GCCUCAACGAGCGGCACUATT | 9 |
| 257 | R: UAGUGCCGCUCGUUGAGGCTT | 10 |
| PGAM2 siRNA- | F: ACUACAAGUCCAUCAGCAATT | 11 |
| 395 | R: UUGCUGAUGGACUUGUAGUTT | 12 |

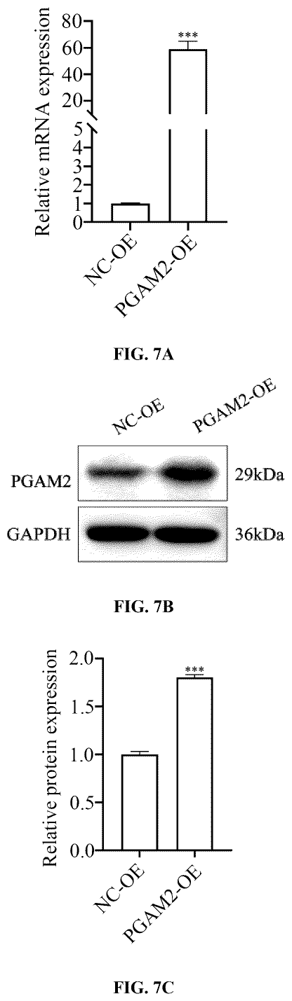

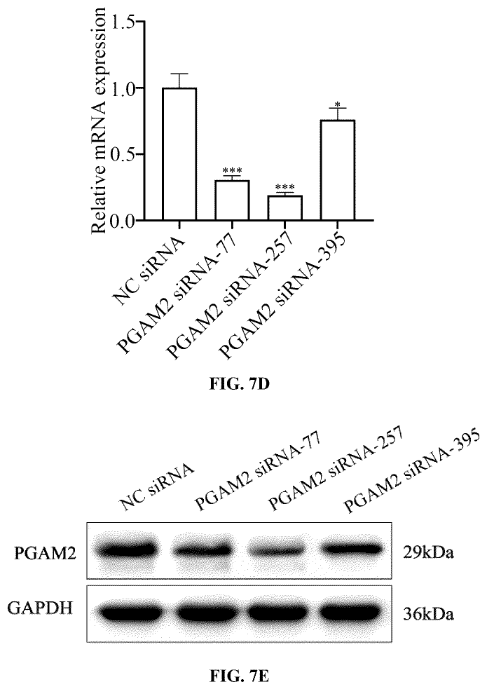

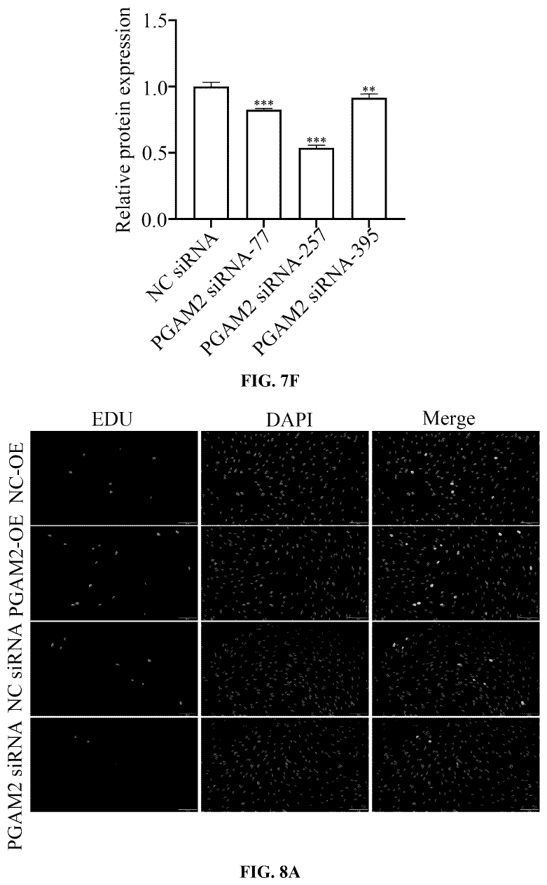

Normally cultured Sertoli cells were taken, and the original medium was aspirated. The cells were washed with phosphate buffered saline (PBS), and digested with trypsin for 3 min, and then the digestion was terminated. The cell suspension was centrifuged at 200 g for 5 min. 20 μL of trypan blue was added to 20 μL of cell suspension for counting. The cells were plated at 3.5×10⁵ cells/well (6-well plate), with 2 mL per well. Afterwards, the cells were cultured at 5% CO₂, 37° C. overnight, and transfected with the overexpression plasmid and the interference plasmids, respectively. Screening was performed to obtain cells in the high PGAM2 expression group and cells in the PGAM2 interference group. After 24 h, the cells were collected for subsequent detection. The fluorescence quantification assay and western blot assay results (FIGS. 7A-7B) showed that compared with the NC-OE group, the PGAM2-OE group had an extremely significantly increased expression level of PGAM2 (P<0.001); and compared with the NC siRNA group, both the PGAM2 siRNA-77 group and the PGAM2 siRNA-257 group had extremely significantly decreased mRNA and protein expression levels of PGAM2 (P<0.001); the PGAM2 siRNA-395 group had a significantly decreased mRNA expression level (P<0.05) and an extremely significantly decreased protein expression level (P<0.01) of PGAM2. This indicates that the Blcattle black cattle Sertoli cells with differential expression of PGAM2 are successfully constructed and can be used for subsequent verification experiments; and the PGAM2 siRNA-257 has the best interference efficiency and can be used for subsequent experiments.

### Example 3. Effect of PGAM2 on Proliferation and Apoptosis of Blcattle Black Cattle Sertoli Cells

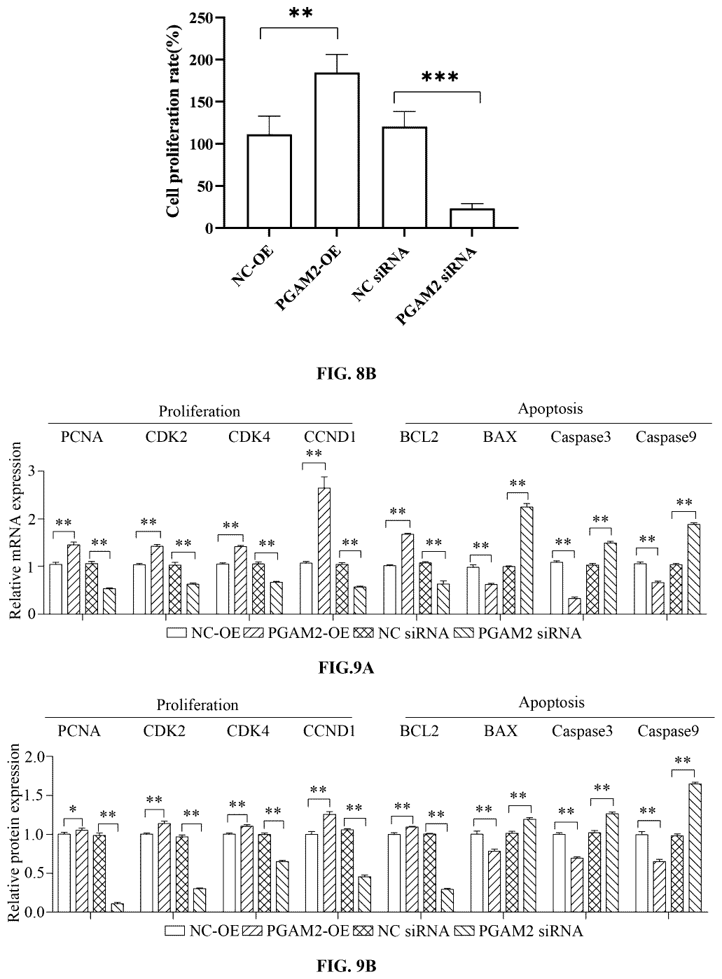

Normally cultured Sertoli cells were taken, and the medium was removed. The cells were rinsed with PBS, and digested with trypsin for 3 min, and then the digestion was terminated. The cell suspension was centrifuged at 200 g for 5 min, and resuspended with a medium. Following this, 20 μL of trypan blue was added to 20 μL of cell suspension for counting. The cells were plated at 45000 cells/well (24-well plate), and 500 μL was added. The cells were cultured at 5% CO₂, 37° C., and then transfected with the overexpression plasmid and the interference plasmids. The cells were incubated with 5-ethynyl-2′-deoxyuridine (EdU) solution for 2 h. After washed with PBS, the cells were incubated with a cell fixative for 30 min, followed by the addition of glycine, and washing with PBS. A permeant was added, followed by another wash with PBS. Apollo staining solution was added, incubation was performed in the dark for 30 min, and the reaction solution was discarded. The permeant was added again, and discarded after washing. Hoechst reaction solution was added, and discarded after incubation in the dark for 30 min. The cells were counterstain with DAPI, sealed, and observed under microscopy to detect EDU positive signals for. data analysis. The EDU results (FIGS. 8A-8B) showed that compared with the NC-OE group, the PGAM2-OE group had an extremely significantly increased cell proliferation rate (P<0.01), indicating a significant increase in cells in S phase; and compared with the NC siRNA group, the PGAM2 siRNA group had an extremely significantly decreased cell proliferation rate (P<0.001), indicating a significant decrease in cells in S phase. These findings clearly demonstrate that PGAM2 promotes the proliferation of Blcattle black cattle Sertoli cells.

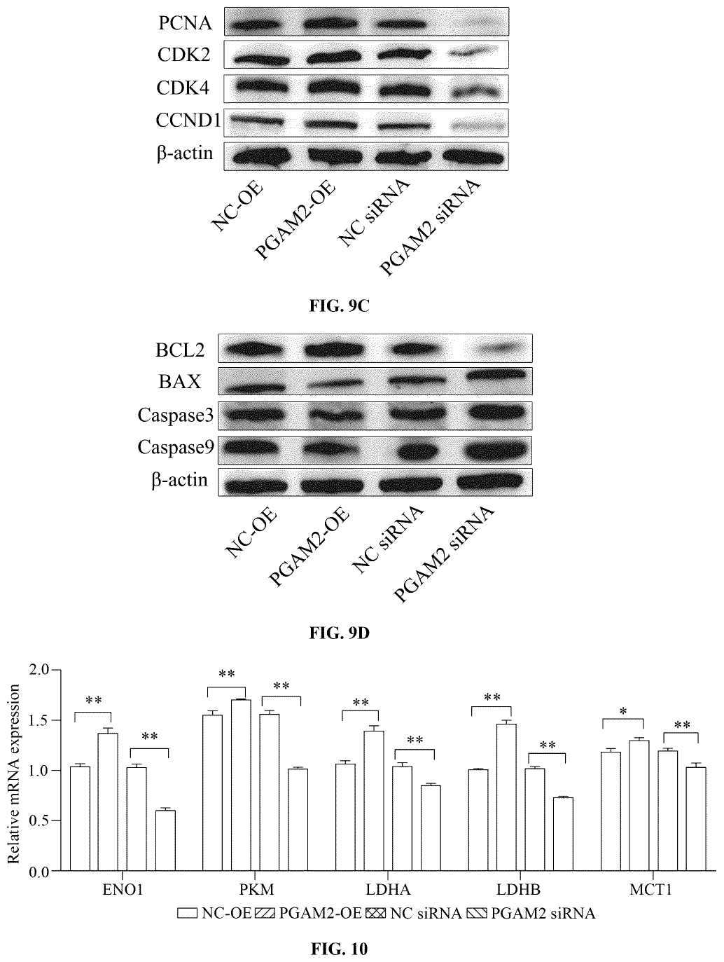

Normally cultured Sertoli cells were transfected with the overexpression plasmid and the interference plasmids. After 48 h of culture in a medium, the RNA and protein were extracted, and the changes in proliferation marker genes (PCNA, CDK2, CDK4, CCND1) and apoptosis marker genes (BCL2, BAX, Caspase3, Caspase9) were detected. The results in FIGS. 9A-9D showed that the PGAM2-OE group had an extremely significantly increased mRNA level (P<0.01) and a significantly increased protein level (P<0.05) of PCNA, the PGAM2-OE group had extremely significantly increased mRNA and protein levels of CDK2, CDK4 and CCND1 (P<0.01), and the PGAM2 siRNA group has extremely significantly decreased mRNA and protein levels of PCNA, CDK2, CDK4 and CCND1 (P<0.01), indicating that PGAM2 promotes the proliferation of Sertoli cells; the PGAM2-OE group had extremely significantly increased mRNA and protein levels of BCL2 (P<0.01), and extremely significantly decreased mRNA and protein levels of BAX, Caspase3 and Caspase9 (P<0.01), and the PGAM2 siRNA group had extremely significantly decreased mRNA and protein levels of BCL2 (P<0.01), and extremely significantly increased mRNA and protein levels of BAX, Caspase3 and Caspase9 (P<0.01), indicating that PGAM2 inhibits the apoptosis of Sertoli cells.

### Example 4 Effect of PGAM2 Gene on Glycolysis in Sertoli Cells

Normally cultured Sertoli cells were transfected with the overexpression plasmid and the interference plasmids. After 48 h of culture in a medium, the RNA was extracted, and the changes in the expression of genes (ENO1, PKM, LDHA, LDHB, MCT1) downstream of glycolysis were detected. The results in FIG. 10 showed that compared with the NC-OE group, the PGAM2-OE group had extremely significantly increased mRNA levels of ENO1, PKM, LDHA and LDHB (P<0.01), and a significantly increased mRNA level of MCT1 (P<0.05); and compared with the NC siRNA group, the PGAM2 siRNA group had extremely significantly decreased mRNA levels of ENO1, PKM, LDHA, LDHB and MCT1 (P<0.01).

### Example 5 Effect of PGAM2 on the Actin Cytoskeleton of Sertoli Cells

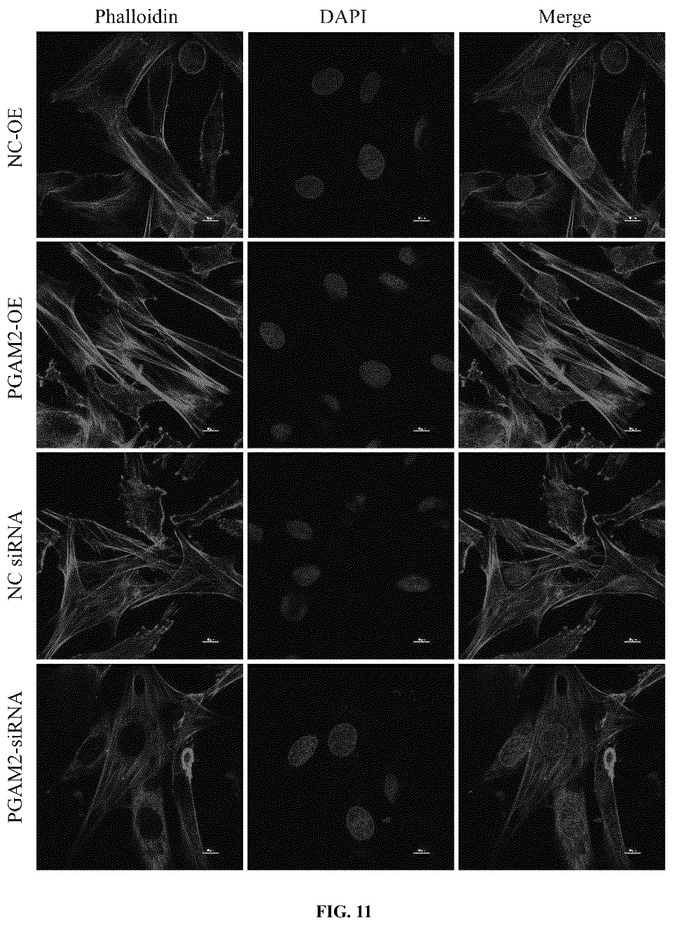

Normally cultured Sertoli cells were transfected with the overexpression plasmid and the interference plasmids, and cultured in a medium for 48 h. Cell fixing and staining: the cells were washed 3 times with PBS and fixed on ice with 4% paraformaldehyde in PBS for 15 min. Following this, the cells were washed 3 times with PBS, permeabilized with 0.2% Triton X-100 in PBS for 5 min at room temperature, and were then washed 3 times with PBS again. Next, a phalloidin stock solution was diluted at a ratio of 1:100; the staining working solution was added dropwise to the slide at a ratio of 250 μL per well, followed by incubation for 20 min at room temperature in the dark; the cells were washed 3 times with PBS. Nuclear staining with 20 μL of DAPI, which was added dropwise to the sealing film, and the slide was taken out and placed on the DAPI, followed by incubation for 10 min at room temperature in the dark; the slide was placed back into the 24-well plate and then washed 3 times with a washing solution, with 3-5 min for each washing. Next, the slide was clamped out, dried by absorbing with an absorbent paper, and sealed with a mounting medium containing an anti-fluorescence quencher for laser confocal fluorescence detection. The phalloidin staining results in FIG. 11 showed that the PGAM2-OE group had a significant increase in microfilament staining and the PGAM2 siRNA group had a significant decrease in microfilament staining; in the PGAM2-OE group, the microfilaments were clear and ordered, and formed a denser network structure, while in the PGAM2 siRNA group, the microfilaments obviously shrunk, the filamentous fibers became shorter and disordered, and the cytoskeleton was disrupted. This indicates that PGAM2 can affect the integrity of the blood-testis barrier and regulate the information communication between Sertoli cells and germ cells by affecting the microfilamentous skeleton.

The descriptions above are exemplary embodiments of the present disclosure and are not used to limit the present disclosure. For those skilled in the art, various changes and modifications can be made to the present disclosure. Any modifications, equivalent replacements, improvements, etc. made without departing from the spirit and principle of the present disclosure shall fall within the scope of the claims of the present disclosure.

What is claimed is:

1. A method for breeding Blcattle black cattle, comprising overexpressing phosphoglycerate mutase 2 (PGAM2) gene in breeding cattle or breeding cattle testis tissue with drugs or genetic engineering methods.

2. The method of claim 1, wherein individuals overexpressing the PGAM2 gene are screened as breeding cattle.

3. An engineered bovine somatic cell, wherein the engineered bovine somatic cell is a bovine Sertoli cell in which PGAM2 gene is knocked down, knocked out, or overexpressed.

4. A method for preparing the engineered bovine somatic cell of claim 3, comprising the steps of:
(1) isolating Sertoli cells from bovine testis tissue;
(2) transfecting the Sertoli cells with a PGAM2 gene overexpression plasmid or a siRNA/shRNA plasmid; and
(3) screening Sertoli cells having up regulated or down regulated PGAM2 gene expression.

5. The method of claim 4, wherein siRNAs used in the method have the nucleotide sequences selected from the group consisting of SEQ ID NOs: 7 and 8, SEQ ID NOs: 9 and 10, and SEQ ID NOs: 11 and 12, respectively.

6. A method for assessing effect of PGAM2 on the proliferation, apoptosis, glycolytic metabolism and actin cytoskeleton of bovine Sertoli cells, comprising administering to a subject the engineered bovine somatic cell of claim 3.

7. A method for screening a drug that targets PGAM2, comprising using the engineered bovine somatic cell of claim 3.


SEQUENCE LISTING (12 sequences)

SEQ ID NO: 1 - DNA, 22 bp, synthetic construct
  source 1..22: mol_type = other DNA; note = forward primer for PGAM2
tttgacatctgctacacatcgg

SEQ ID NO: 2 - DNA, 23 bp, synthetic construct
  source 1..23: mol_type = other DNA; note = reverse primer for PGAM2
acttgtagtaggggtgtttctca

SEQ ID NO: 3 - DNA, 20 bp, synthetic construct
  source 1..20: mol_type = other DNA; note = forward primer for GAPDH
gatgctggtgctgagtatgt

SEQ ID NO: 4 - DNA, 20 bp, synthetic construct
  source 1..20: mol_type = other DNA; note = reverse primer for GAPDH
gcagaaggtgcagagatgat

SEQ ID NO: 5 - DNA, 21 bp, synthetic construct
  source 1..21: mol_type = other DNA; note = sense strand of NC siRNA
  misc_feature 1..19: note = RNA
  misc_feature 20..21: note = DNA
ttctccgaacgtgtcacgttt

SEQ ID NO: 6 - DNA, 22 bp, synthetic construct
  source 1..22: mol_type = other DNA; note = antisense strand of NC siRNA
  misc_feature 1: note = DNA
  misc_feature 21..22: note = DNA
  misc_feature 2..20: note = RNA
tacgtgacacgttcggagaatt

SEQ ID NO: 7 - DNA, 21 bp, synthetic construct
  source 1..21: mol_type = other DNA; note = sense strand of PGAM2 siRNA-77
  misc_feature 1..19: note = RNA
  misc_feature 20..21: note = DNA
gatgcagagctgagcgagatt

SEQ ID NO: 8 - DNA, 21 bp, synthetic construct
  source 1..21: mol_type = other DNA; note = antisense strand of PGAM2 siRNA-77
  misc_feature 1..19: note = RNA
  misc_feature 20..21: note = DNA
tctcgctcagctctgcatctt

SEQ ID NO: 9 - DNA, 21 bp, synthetic construct
  source 1..21: mol_type = other DNA; note = sense strand of PGAM2 siRNA-257
  misc_feature 1..19: note = RNA
  misc_feature 20..21: note = DNA
gcctcaacgagcggcactatt

SEQ ID NO: 10 - DNA, 21 bp, synthetic construct
  misc_feature 1..19: note = RNA
  source 1..21: mol_type = other DNA; note = antisense strand of PGAM2 siRNA-257
  misc_feature 20..21: note = DNA
tagtgccgctcgttgaggctt

SEQ ID NO: 11 - DNA, 21 bp, synthetic construct
  source 1..21: mol_type = other DNA; note = sense strand of PGAM2 siRNA-395
  misc_feature 1..19: note = RNA
  misc_feature 20..21: note = DNA
actacaagtccatcagcaatt

SEQ ID NO: 12 - DNA, 21 bp, synthetic construct
  misc_feature 1..19: note = RNA
  source 1..21: mol_type = other DNA; note = antisense strand of PGAM2 siRNA-395
  misc_feature 20..21: note = DNA
ttgctgatggacttgtagttt